SEQUENCE LISTING (6 sequences)

SEQ ID NO: 1 - DNA, 20 bp, Artificial Sequence
  Primer
agcaggcaaacctggtgaac

SEQ ID NO: 2 - DNA, 20 bp, Artificial Sequence
  Primer
aacctctctcgcctcttgct

SEQ ID NO: 3 - DNA, 23 bp, Artificial Sequence
  Primer
agcagctgccgaagtcagttcct

SEQ ID NO: 4 - DNA, 24 bp, Artificial Sequence
  Primer
gttctgacatggcgcctcctctga

SEQ ID NO: 5 - DNA, 18 bp, Artificial Sequence
  Primer
gaaggtgaaggtcggagt

SEQ ID NO: 6 - DNA, 20 bp, Artificial Sequence
  Primer
gaagatggtgatgggatttc

(19) United States
(12) Patent Application Publication
(10) Pub. No.: US 2021/0338568 A1
(43) Pub. Date: Nov. 4, 2021

(54) COSMETIC METHOD

(71) Applicant: Shiseido Company, Ltd., Chuo-ku, Tokyo (JP)

(72) Inventor: Tomonobu EZURE, Tokyo (JP)

(73) Assignee: Shiseido Company, Ltd., Chuo-ku, Tokyo (JP)

(21) Appl. No.: 17/271,969

(22) PCT Filed: Aug. 27, 2019
(86) PCT No.: PCT/JP2019/033554

§ 371 Date: Feb. 26, 2021

(30) Foreign Application Priority Data
Aug. 31, 2018 (JP) 2018-164004

(51) Int. Cl. A61K 8/98; A61K 35/36; A61L 27/60; A61Q 19/00; A61H 39/04; A61H 7/00

(52) U.S. Cl. CPC A61K 8/981; A61K 35/36; A61H 7/00; A61Q 19/00; A61H 39/04; A61L 27/60

(57) ABSTRACT

This invention provides a method of causing proliferation of stem cells and their derivative cells and thus solves the cosmetic problems of wrinkles and sagging. Mechanical stress is applied to skin that includes sebaceous glands, thereby causing proliferation of stem cells and their derivative cells.

### FIELD

The present invention relates to a method for increasing stem cells and their derivative cells, and to a cosmetic method utilizing the method.

### BACKGROUND

The dermis is composed mainly of interstitial components and cell components, and also includes vessels and nerves. The dermis is constantly subjected to external factors such as temperature changes, ultraviolet irradiation and physical irritation, and internal factors such as stress and aging. This results in changes in the cell activity of dermal fibroblasts within the dermis layer. Dermal fibroblasts produce interstitial components associated with skin elasticity and tightness, and therefore lower cell activity results in thinning of the dermis layer, loss of elasticity and wrinkles or sagging, which are major cosmetic problems. Much research has been carried out to date on components that inhibit the activity of interstitial component-decomposing enzymes such as heparanases and matrix metalloproteinases, as chemical agents for improving wrinkles, sagging and tightness (PTL 1: Japanese Unexamined Patent Publication No. 2016-169238, PTL 2: Japanese Unexamined Patent Publication No. 2012-144499). Components that increase cellular activity of dermal fibroblasts are also being studied, and several dermal fibroblast activators have been developed (PTL 3: Japanese Unexamined Patent Publication No. 2006-262806).

### CITATION LIST

### Patent Literature

[PTL 1] Japanese Unexamined Patent Publication No. 2016-169238

[PTL 2] Japanese Unexamined Patent Publication No. 2012-144499

[PTL 3] Japanese Unexamined Patent Publication No. 2006-262806

### SUMMARY

### Technical Problem

Research by the present inventors has shown that once the cell activity of dermal fibroblasts has been reduced due to aging or other reasons, it becomes difficult to restore the cell activity. It has been concluded that the cosmetic problem of wrinkles and sagging is more effectively solved by inducing differentiation of new dermal fibroblasts, rather than promoting the cell activity of dermal fibroblasts whose cell activity has already been reduced.

### Solution to Problem

As a result of avid research on methods of inducing differentiation from stem cells to dermal fibroblasts, the present inventors have found, surprisingly, that division of stem cells is promoted and new dermal fibroblasts are induced by mechanical stress of the areas around sebaceous glands which contain large amounts of stem cells.

The present invention relates to the following:

[1] A method of causing proliferation of stem cells or their derivative cells around sebaceous glands, comprising applying mechanical stress to a skin sample that includes sebaceous glands which has been harvested from skin, during organ culture of the skin sample.

[2] The method according to [1] above, wherein the mechanical stress is stretching stress.

[3] A cosmetic method, comprising applying mechanical stress to skin for which sagging, wrinkles or tightness is a concern, thus causing proliferation of stem cells around sebaceous glands, or their derivative cells.

[4] The cosmetic method according to [3] above, wherein the sebaceous gland density in the skin is a density of 10 to 80% of the skin region.

[5] The cosmetic method according to [3] or [4] above, wherein the mechanical stress is stretching, compression or suction of the skin.

[6] A cosmetic device comprising:

a measuring unit that measures sebaceous gland density, and

a mechanical stress applicator,

wherein the cosmetic device applies mechanical stress to a skin region with high sebaceous gland density, causing proliferation of stem cells around the sebaceous glands, or their derivative cells.

### BRIEF DESCRIPTION OF DRAWINGS

FIG. 1 is a pair of photographs showing remodeling of fibroblasts in the dermis of young and aged subjects, as observed under a three-dimensional electron microscope (SBF-SEM).

FIG. 2 shows a pair of photographs taken of a sample of cheek skin from a young subject (age 20) and cheek skin from an aged subject (age 80), after tissue staining.

FIG. 3 shows a pair of photographs taken in a cheek skin sample from an aged subject (age 80), after staining of the stem cells in the major dermal layer and around the sebaceous gland.

FIG. 4A is pair of photographs taken of skin samples after staining of CD54-positive stem cells, one after organ culture without application of stretching stress (control), and the other after organ culture with application of stretching stress. FIG. 4B is pair of photographs taken of skin samples after staining of collagen, one after organ culture without application of stretching stress (control), and the other after organ culture with application of stretching stress.

FIG. 5A shows a pair of photographs of the nasolabial groove before and after applying stretching stress to the cheek. FIG. 5B is a graph showing improvement in nasolabial groove wrinkle by stretching stress.

FIG. 6 is a set of photomicrographs taken after 2 days of culturing of 2.5 ml of cell suspensions prepared to 0.25×10⁴ cell/ml, 0.5×10⁴ cell/ml, 1.0×10⁴ cell/ml, 2.0×10⁴ cell/ml and 4.0×10⁴ cell/ml, showing the degree of contact at each concentration.

FIG. 7 is a graph showing a comparison of the expression level of type I collagen at each degree of contact.

FIG. 8 shows the inhibiting effect on gene expression by siRNA for each gene.

FIG. 9 shows the change in expression level of type I collagen when the gene expression was inhibited by siRNA for each gene.

FIG. 10 shows the cell proliferation rate after inhibiting gene expression of cadherin 2 (A) and gene expression of p21 (B), for cultured dermal fibroblasts.

FIG. 11 shows shape deformation after inhibiting gene expression of cadherin 2 in cultured dermal fibroblasts. Knockout of cadherin 2 eliminated visible intercellular adhesion and caused the shapes to become rounded, while also resulting in visible staining of the aged marker n-Gal.

### DESCRIPTION OF EMBODIMENTS

The present invention relates to a method of causing proliferation of stem cells or their derivative cells. The method includes a step of applying mechanical stress to a skin sample containing sebaceous glands, which has been harvested from skin, when the skin sample is cultured by organ culture.

The region around sebaceous glands includes stem cells that are able to differentiate to dermal fibroblasts. These stem cells are characterized by CD54 expression. When a skin sample containing sebaceous glands is cultured by organ culture with application of mechanical stress to the skin sample, the stem cells around sebaceous glands increase in number. The increased cells may be those maintaining their sternness, or may be derivative cells that have been differentiated from the stem cells. Derivative cells differentiated from stem cells include dermal progenitor cells and dermal fibroblasts. The stem cells present in the dermis may be referred to as dermal stem cells since they are able to differentiate into dermal fibroblasts.

Mechanical stress applied to a skin sample or skin is a form of physical stimulation that produces a mechanical effect. Mechanical stress is not limited to stress applied through a machine such as a probe or actuator, and may be applied by any means that can produce a mechanical effect. Examples of mechanical stress include one or more from among compression, suction, compaction and stretching. The mechanical stress may be applied in the direction parallel to the surface of the skin sample or skin, i.e. laterally, or the mechanical stress may be applied in the direction perpendicular to the surface, i.e. vertically. The strength of the mechanical stress may be set as desired in a range such that the skin sample or skin is not damaged, ruptured or destroyed. A preferred example is application of compaction or stretching stress to a skin sample to produce about 10% to 50% deformity of the skin sample. The deformation produced is preferably 20 to 40% and more preferably about 30%.

Sebaceous glands are sebum-producing organs found accompanying hair follicles. Sebaceous glands are distributed in general throughout the body except for the palms of the hands and the soles of the feet. The skin sample for the invention may therefore be obtained from any part of the skin, and in particular it may be obtained from skin at seborrhea sites where sebaceous glands have developed. Seborrhea sites include the forehead, ala nasi, nasolabial groove, scalp, sternum, underarms, abdominal region and vulva. According to one mode, a site may be selected for harvesting of a sample, based on the proportion of the region of sebaceous glands occupying the epidermis. One example would be to obtain a sample of skin where sebaceous glands occupy 10 to 80% of the skin region. The sebaceous gland region is preferably 30 to 80% and more preferably 50 to 80%. The size of the skin sample may be selected as desired, such as a slice with a surface area of 100 mm² to 10,000 mm², for example, and a depth up to the dermis layer. From the viewpoint of minimal invasiveness, it is preferably 500 mm² or smaller and more preferably 300 mm² or smaller. From the viewpoint of obtaining a sufficient amount of stem cells or their derivative cells, on the other hand, it is also preferably 100 mm² or larger and more preferably 500 mm² or larger.

Organ culture of the skin sample may be carried out by an established method, such as the method described in Journal of Dermatological Science 74 (2014)236-241, for example. Mechanical stress, and especially stretching stress, can be applied before culturing or during culturing. The strength of the mechanical stress may be selected as appropriate for the size of the skin sample. One example is stretching stress applied by pulling with the ends of the skin sample anchored at opposite ends with forceps. The mechanical stress may be applied for the entirety or only a portion of the organ culture period.

The stem cells or their derivative cells grown in the organ culture step may be isolated or separated by cutting or enzymatic treatment of the cultured organ. The separated cells may be injected into the dermis layer of interest, either after further culturing or directly without culturing.

According to another mode, the present invention relates to a cosmetic method that involves proliferation of stem cells around sebaceous glands, or their derivative cells, by application of mechanical stress to skin. In such a cosmetic method, the stem cells around sebaceous glands, or their derivative cells proliferate, with the cells differentiating to dermal progenitor cells and dermal fibroblasts. The cosmetic method of the invention can therefore promote increase in the number of dermal fibroblasts in the dermis, thereby accelerating production of interstitial components. The cosmetic method of the invention may thus be considered to be a method of promoting production of interstitial components, or a method of improving skin wrinkles, sagging or tightness.

The subject for application for the cosmetic method of the invention may be any subject who is concerned about wrinkles or sagging, or who has reduced skin tightness. Sagging can be evaluated using direct visual assessment. The cosmetic method of the invention is preferably applied for values of 6 or lower, preferably 5 or lower and more preferably 4 or lower in measurement by visual assessment, and/or Ur/Uf values of 0.8 or lower, preferably 0.7 or lower and more preferably 0.6 or lower in measurement using a Cutometer.

Mechanical stress applied to skin includes one or more from among compression, suction, compaction and stretching. The mechanical stress applied to skin may be applied in the direction parallel to the skin surface, i.e. laterally, or it may be applied in the direction perpendicular to the skin surface, i.e. vertically. The mechanical stress will usually be applied over a period of 1 minute or longer, preferably 3 minutes or longer and more preferably 5 minutes or longer. While the upper limit is not particularly restricted, it is preferably no longer than 1 hour, more preferably no longer than 30 minutes and even more preferably no longer than 15 minutes, from the viewpoint of ensuring convenience of the method. A device equipped with a member such as a probe or actuator in a manner that applies mechanical stress, may be used to apply the mechanical stress to the skin. As an example of such a device, the actuator described in Japanese Patent Public Inspection No. 2011-505897 may be used. According to another mode, the cosmetic method may include exercising or massaging of the face. Exercising of the face may be inflation of the cheeks or wide opening of the eyes. Massaging of the face may be massage using hands or a roller.

The cosmetic method of the invention can accelerate proliferation of stem cells around sebaceous glands, or their derivative cells. Before and after application of the mechanical stress, therefore, an additional step may be included in which the stem cells around the sebaceous glands, or their derivative cells, are observed. Following application of the mechanical stress, the stem cells or their derivative cells are preferably observed in an immunohistochemical manner after elapse of 24 hours or longer and preferably 48 hours or longer.

Instead of observation of the stem cells or their derivative cells, a step of improvement in wrinkles, sagging and tightness may be included. A publicly known measuring instrument (such as described in Japanese Unexamined Patent Publication No. 2017-064391) may be used to measure improvement in wrinkles or sagging. A Dermal Torque Meter or Cutometer may be used for measurement of skin tightness.

Mechanical stress according to the invention is preferably applied to a region with high sebaceous gland density. Applying mechanical stimulation to a region with a sebaceous gland density of preferably 200/cm² or greater and more preferably 400/cm² or greater is preferred from the viewpoint of causing proliferation of stem cells around sebaceous glands, or their derivative cells.

Another mode of the invention relates to a cosmetic device comprising:

a sebaceous gland detector, and

a mechanical stress applicator,

wherein the cosmetic device applies mechanical stress to a skin region with high sebaceous gland density, causing proliferation of stem cells around the sebaceous glands, or their derivative cells.

The sebaceous gland detector may be a microscope, for example, which can detect the locations and number of sebaceous glands. Proliferation of stem cells around sebaceous glands, or their derivative cells, can be accelerated in a region with a high density of sebaceous glands by application of mechanical stress with a mechanical stress applicator.

Examples of mechanical stress include one or more from among compression, suction and stretching. The mechanical stress applicator is equipped with a member such as a probe or actuator in a manner that applies such mechanical stress. A pressing probe can apply mechanical stress to the skin by pushing into the skin in the vertical direction with respect to the skin. A suction probe can apply mechanical stress to skin by contacting the skin and applying negative pressure. A stretching probe causes one or more contact sites where it contacts with the skin to move in the horizontal direction with respect to the skin surface, thus allowing mechanical stress to be applied to the skin.

Dermal fibroblasts are fibroblasts present in the dermis, being cells that produce interstitial components in the dermis. Dermal fibroblasts induced from stem cells are therefore useful for supplementing dermal interstitial components that have been reduced due to external factors or internal factors. Accelerated collagen production has actually been observed near stem cells around sebaceous glands that have been increased by mechanical stress (FIG. 4B).

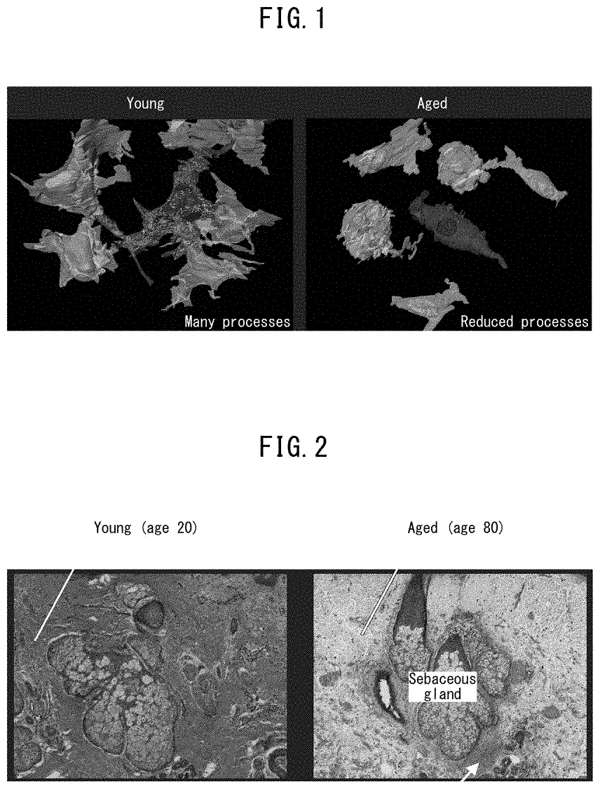

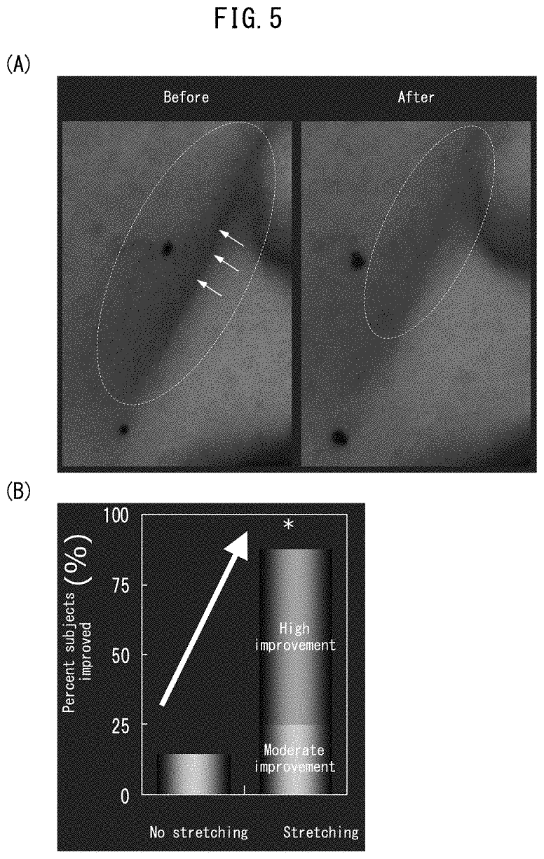

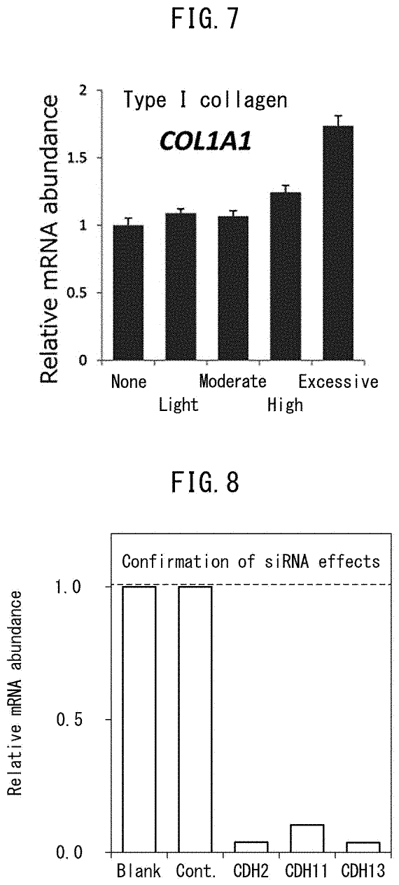

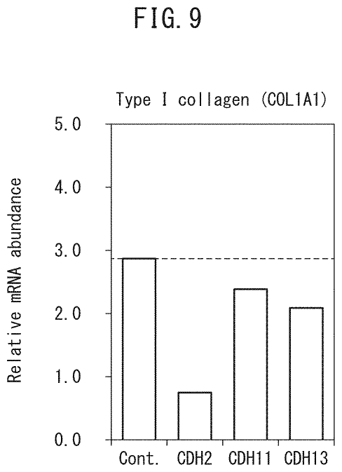

Research by the present inventors has also shown that dermal fibroblasts in the dermis of elderly persons differs in form from dermal fibroblasts present in the dermis of young persons, and that the latter dermal fibroblasts are in mutual adhesion with each other, in contradiction to previous knowledge (FIG. 1). Specifically, the dermal fibroblasts of young persons have processes that adhere to other fibroblasts, while the fibroblasts of elderly persons are spherical with few processes and have less or no adhesion to other fibroblasts. It is believed that it is the lost intercellular adhesion by the dermal fibroblasts of elderly persons that causes reduction in cell activity including production of interstitial components. Research by the present inventors has also shown that production volume of collagen varies depending on the intercellular adhesion of dermal fibroblasts FIG. 7), that cadherin 2 is involved in the intercellular adhesion (FIG. 9), and that intercellular adhesion contributes to the ability to produce interstitial components including collagen, i.e. to cell activity. While it is not our intention to be limited to any particular theory, it is thought that stem cells around sebaceous glands that have increased in number due to mechanical stress according to the invention differentiate to dermal fibroblasts as they proliferate. Since this results in a greater number of surrounding dermal fibroblasts, intercellular adhesion also increases and consequently dermal fibroblasts with high cell activity increase around the sebaceous glands, thus leading to higher collagen production (FIG. 4B), which presumably is responsible for the actual improvement in wrinkles that is seen in vivo (FIG. 5).

Chemical agents have been sought in the past that are able to activate dermal fibroblasts, for the purpose of beautification, and especially for improving wrinkles, sagging and tightness, and many components including extracts have been selected. However, with fibroblasts that have lost their processes and no longer have cellular adhesion, it may be difficult to regain adhesion. The present inventors therefore considered stimulating stem cells that are present as suppliers of dermal fibroblasts, instead of activating already existing dermal fibroblasts, and found that stem cells around sebaceous glands, or their derivative cells can be increased in number by application of mechanical stress (FIG. 4). The dermal fibroblasts that have been newly induced from stem cells have processes and can form adhesion with other fibroblasts. By adhering with other fibroblasts, the dermal fibroblasts newly induced from stem cells have excellent ability to produce interstitial components such as collagen (FIG. 7).

Interstitial components in the dermis consist mainly of collagen fibers, elastic fibers and matrix substances. Accelerating the cell activity of dermal fibroblasts, therefore, increases the production of at least one, preferably 2 and more preferably all of the components from among collagen fibers, elastic fibers and matrix substances.

Collagen fibers are composed of collagen. About 20 different collagen molecules are known, based on differences in the molecular structure of the a chain. According to the invention, the collagen may be any type of collagen but is preferably type I collagen, type III collagen, type V collagen, type IV, type VII or type 17 collagen, which are found mainly in the dermis, more preferably type I collagen, type III collagen or type V collagen, and most preferably type I collagen, which occupies about 80% of the dermis. The amount of collagen fiber production can be determined by measuring collagen production or expression levels. The collagen production level varies according to the degree of dermal fibroblast adhesion. Since adhesion between cells is maintained with fibroblasts newly induced from stem cells even in the skin of elderly persons, production of interstitial components including collagen is high.

The main component of elastic fibers is elastin, with fibrillin being wound around the elastin. The elastic fiber production level can be determined by measuring the production or expression level of either or both elastin or fibrillin.

The matrix is composed mainly of glycoproteins or proteoglycans, forming the extracellular matrix. Glycoproteins are sugar-containing proteins and include fibronectin which is found in the dermis and which binds with cell surface proteins to function as a cell scaffold, while also binding with other polymers such as collagen. Proteoglycans are macromolecules having a glycosaminoglycan bound to a core protein, with hyaluronic acid and dermatan sulfate being included as the major glycosaminoglycans in the dermis. Proteoglycans function primarily to help retain moisture in the dermis.

All of the publications mentioned throughout the present specification are incorporated herein in their entirety by reference.

The examples of the invention described below are intended to serve merely as illustration and do not limit the technical scope of the invention. The technical scope of the invention is limited solely by the description in the Claims. Modifications of the invention, such as additions, deletions or substitutions to the constituent features of the invention, are possible so long as the gist of the invention is maintained.

### EXAMPLES

Observation with 3D Microscope

Skin slices from a young subject (age 20) and an aged subject (age 80) were treated with a conductive resin and provided for observation with a 3D electron microscope (SBF-SEM). A three-dimensional remodeling photograph is shown (FIG. 1). Sample preparation and observation by SBF-SEM were as follows.

### Serial Block-Face Scanning Electron Microscope (SBF-SEM)

A human skin sample was fixed in buffer solution containing 2% glutaraldehyde and 2% paraformaldehyde for several days at 4° C. The sample was treated for 1 hour at 4° C. with phosphate-buffered physiological saline containing 2% osmium tetroxide (Nisshin EM, Tokyo, Japan) and 1.5% potassium ferrocyanide (Wako Pure Chemical Industries, Ltd.), and then treated for 20 minutes at room temperature with 1% thiocarbohydrazide (Sigma, St. Louis, Mo., USA), treated for 30 minutes at room temperature with a 2% osmium tetroxide aqueous solution, and treated for at least 12 hours at 4° C. and for 1 hour at room temperature with a 2% uranil acetate solution. The sample was then incubated for 30 minutes at 65° C. in a solution containing 0.67% lead nitrate (pH 5.0 to 5.5, TAAB, Berkshire, UK) and 0.03 mL aspartic acid (Nacalai Tesque, Kyoto, Japan). The sample was dehydrated in a stepwise ethanol series, treated with dehydrated acetone, permeated with Queto1812 epoxy resin (Nisshin EM, Tokyo, Japan) at 35° C., and embedded in Kejen black powder-containing Queto1812 (Nguyen et al. Sci Rep. 2016). The resin was incubated for longer than 7 nights at 70° C., and polymerization was confirmed. Observation was made by SBF-SEM using a field emission scanning electron microscope (Merlin, Carl Zeiss, Oberkochen, Germany) equipped with a 3View chamber Ultramicrotome system (Gatan, Pleasanton, Calif.). A continuous image sequence was obtained at 80 to 90 μm×80 to 90 μm width (11 to 12 nm/pixel), with a step of 80 nm over a depth of 60 μm. The continuous images were processed using FIJI (https://fiji.sc/). Segmentation and 3D reconstruction were carried out using an Amira (Maxnet Co., Ltd, Tokyo, Japan).

### Observation of Skin Slices

A skin slice obtained from the cheek of a young person and a skin slice obtained from the cheek of an elderly person were each van Gieson-stained and observed under a microscope (FIG. 2). Regions containing sebaceous glands were photographed (FIG. 2). In the young female, the staining intensity was high indicating a high abundance of collagen in the dermis layer, whereas in the elderly female, the staining intensity was low in the dermis layer, indicating a low amount of collagen. Large amounts of collagen were found in the regions surrounding the sebaceous glands (white arrows).

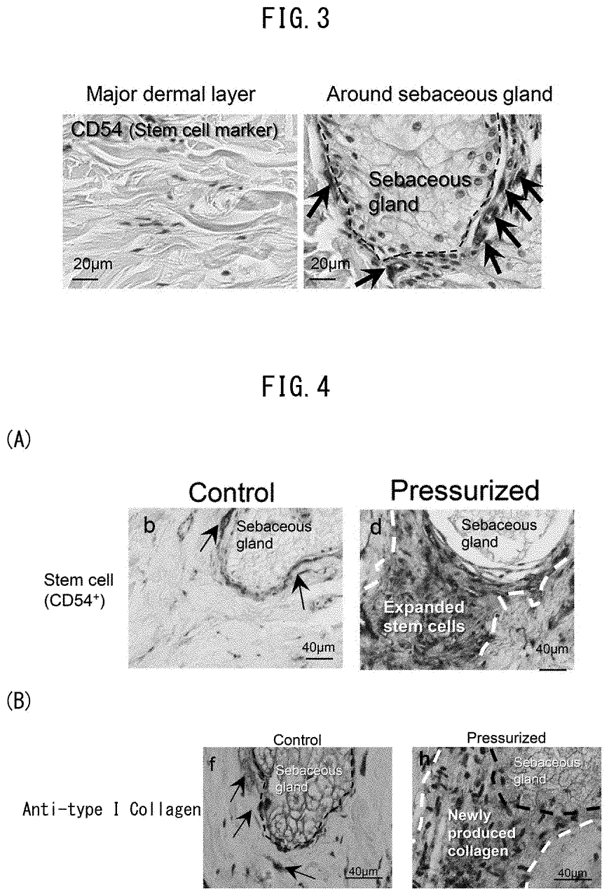

The skin slice obtained from the cheek of the elderly person was fixed with acetone and reacted with anti-CD54 antibody, and the stem cells were then identified using EnVision (DAKO). Separate photographs were taken of the whole dermis and the regions around the sebaceous glands. Although stem cells were also present in the skin of the elderly person, the amount was very low in the whole dermis, with more stem cells present in the areas surrounding the sebaceous glands (FIG. 3: Black dotted lines indicate the boundaries of the sebaceous glands, and arrows indicate stem cells).

The experiment suggested that dermal fibroblasts present in elderly dermis have lost the ability to adhere to other cells and thus have reduced cell activity (collagen production). It also suggested that stem cells are abundantly present in the areas surrounding sebaceous glands even in elderly skin, and can serve as suppliers of dermal fibroblasts to contribute to increased collagen production around the sebaceous glands.

### Organ Culture Experiment

Two 10 mm² human skin sample slices were obtained. One of the slices was cultured without application of compaction stress while the other was subjected to vertical force so as to be deformed about 30% in the vertical direction, and was immersed in 10% FBS-containing DMEM medium and cultured for 7 days in a 5% CO₂, 37° C. atmosphere.

The cultured skin sample slices were fixed with acetone and the stem cells were visualized with an EnVision (DAKO), using anti-CD54 antibody. The results are shown in FIG. 4A. Stem cells or cells induced from stem cells were observed to have increased in number in the skin sample subjected to compaction stress. The cultured skin sample slices were also fixed with acetone and reacted with anti-type I collagen antibody and visualized using EnVision (DAKO). The results are shown in FIG. 4B. They show that collagen production was increased around the sebaceous glands in the skin sample subjected to compaction stress.

### Effect of Mechanical Stress

Eight female subjects (age 40) exercised by inflating the cheeks once per day for 10 minutes, to apply stretching stress to the cheeks. The experiment was conducted over a period of 2 months, taking photographs (FIG. 5A) and visually evaluating the degree of sagging of the cheeks (FIG. 5B) before the experiment and at a point after 1 month of the experiment.

Improvement was seen from before and after the experiment.

The experiment results indicated that applying mechanical stress such as stretching stress or compaction stress to skin containing sebaceous glands causes proliferation of stem cells or derivative cells from stem cells, and that stretching stress actually contributes to improvement in wrinkles even in vivo.

### Culturing Experiment

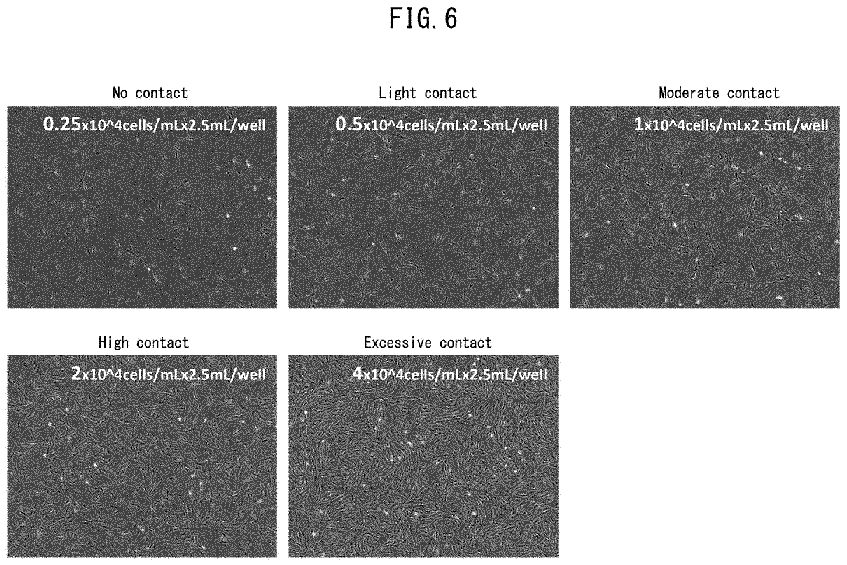

Human primary cultured fibroblasts were obtained from a human skin sample by an established method. The cell count was measured, and 0.25×10⁴ cells (no contact), 0.5×10⁴ cells (light contact), 1.0×10⁴ cells (moderate contact), 2.0×10⁴ cells (high contact) and 4.0×10⁴ cells (excessive contact) were prepared per 1 ml of DMEM medium (containing 10% FBS). The cell suspensions were dropped at 2.5 ml each into a 6-well plate (Falcon Co.) and cultured for 2 days in a 37° C., 5% CO₂ atmosphere. FIG. 6 shows photomicrographs of the cultured cells.

### Change in Gene Expression Due to Difference in Adhesion Degree

When the cells were collected from the cultured products with different degrees of contact and subjected to microarray analysis, it was found that type I collagen expression varied depending on the degree of contact of the cells (data not shown). The type I collagen expression levels were determined by real-time PCR using the primers shown below (FIG. 7). GAPDH gene expression was used as an internal standard to determine the expression levels.

TABLE 1

| Primer | Sequence |
|---|---|
| Col1A1 Forward Primer | AGCAGGCAAACCTGGTGAAC |
| | (SEQ ID NO: 1) |
| Col1A1 Reverse Primer | AACCTCTCTCGCCTCTTGCT |
| | (SEQ ID NO: 2) |
| GAPDH Forward Primer | GAAGGTGAAGGTCGGAGT |
| | (SEQ ID NO: 5) |
| GAPDH Reverse Primer | GAAGATGGTGATGGGATTTC |
| | (SEQ ID NO: 6) |

The experiment showed that change in degree of adhesion causes variation in expression of the interstitial component collagen, according to the degree of intercellular adhesion.

### Identification of Adhesion Factors Contributing to Adhesion and Affecting Collagen Expression

The expression of cell adhesion proteins in cultured fibroblasts was inhibited by an siRNA method in order to identify factors contributing to expression of type I collagen. CDH2, CDH11 and CDH13 siRNA were acquired from Qiagen Inc. The expression of each gene in cultured dermal fibroblasts was suppressed using each siRNA according to an established method. After seeding 0.5 ml of cell suspension at a density of 1×10⁴ cell/ml in each well of a 24-well plate (area: 2 cm²), culturing was carried out for 2 days in a 37° C., 5% CO₂ atmosphere. The cultured fibroblasts were harvested and the gene expression of each cell adhesion protein was determined by real-time PCR, confirming suppression of the expression of the protein of interest (FIG. 8). Measurement of type I collagen expression in the harvested cells showed a reduction in type I collagen expression level when CDH2 gene expression was suppressed (FIG. 9).

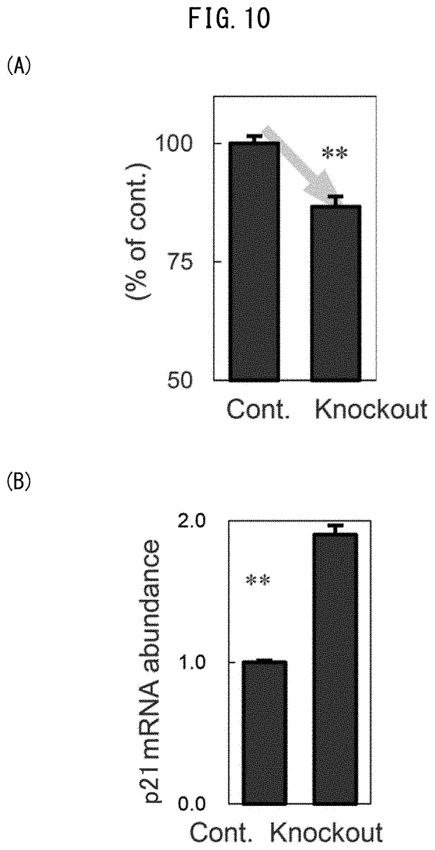

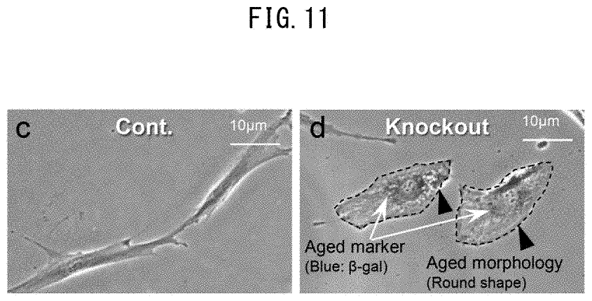

The dermal fibroblasts with inhibited cadherin 2 expression and the control dermal fibroblasts were seeded into each well of a 24-well plate (area: 2 cm²) with 0.5 ml of cell suspension at a density of 1×10⁴ cell/ml each, and were cultured for 2 days in a 37° C., 5% CO₂ atmosphere. The cultured cell counts were measured and the cell proliferations were compared (FIG. 10A). Gene expression of the CDK family inhibitor protein p21 was measured for each of the cultured cells by RT-PCR using the primers shown below (FIG. 10B). A cell senescence assay kit (Biovision) was also used for staining of the cells with X-Gal based on β-galactosidase activity, and the cells were photographed under a microscope (FIG. 11). Among the control cells, the cultured dermal fibroblasts had flat shapes and showed intercellular adhesion, but knockout of cadherin 2 gene expression reduced intercellular adhesion and consequently resulted in more spherical cell shapes than the control. The dermal fibroblasts with inhibited cadherin 2 gene expression had high activity of intracellular β-galactosidase, as a marker of cell senescence.

TABLE 2

| Primer | Sequence |
|---|---|
| P21 Forward Primer | AGCAGCTGCCGAAGTCAGTTCCT |
| | (SEQ ID NO: 3) |
| P21 Reverse Primer | GTTCTGACATGGCGCCTCCTCTGA |
| | (SEQ ID NO: 4) |
| GAPDH Forward Primer | GAAGGTGAAGGTCGGAGT |
| | (SEQ ID NO: 5) |
| GAPDH Reverse Primer | GAAGATGGTGATGGGATTTC |
| | (SEQ ID NO: 6) |

In the dermal fibroblasts with inhibited cadherin 2 gene expression, collagen gene expression and cell proliferation were both suppressed. The dermal fibroblasts with inhibited cadherin 2 gene expression also exhibited increased gene expression of p21 which halts the cell cycle by cyclin inhibition, and further exhibited high intracellular β-galactosidase activity, as a marker of cell senescence. These results indicated that dermal fibroblasts form contact between cells via the cell adhesion protein cadherin 2 and that loss of the contact caused the cells to age and have reduced cell activity.

1. A method of causing proliferation of stem cells around sebaceous glands, or their derivative cells, comprising applying mechanical stress to a skin sample that includes sebaceous glands which has been harvested from skin, during organ culture of the skin sample.

2. The method according to claim 1, wherein the mechanical stress is stretching stress.

3. A cosmetic method comprising applying mechanical stress to skin for which sagging, wrinkles or tightness is a concern, thus causing proliferation of stem cells around sebaceous glands, or their derivative cells.

4. The cosmetic method according to claim 3, wherein the sebaceous gland density in the skin is a density of 10 to 80% of the skin region.

5. The cosmetic method according to claim 3, wherein the mechanical stress is stretching, compression or suction of the skin.

6. A cosmetic device comprising:
a measuring unit that measures sebaceous gland density, and
a mechanical stress applicator,
wherein the cosmetic device applies mechanical stress to a skin region with high sebaceous gland density, causing proliferation of stem cells around the sebaceous glands, or their derivative cells.

7. The cosmetic method according to claim 4, wherein the mechanical stress is stretching, compression or suction of the skin.
